(19) United States
(12) Patent Application Publication
(10) Pub. No.: US 2026/0240807 A1
(43) Pub. Date: Aug. 20, 2026

(54) UROLITHINS FOR IMPROVING CARDIAC FUNCTION AND HEALTH

(71) Applicant: Amazentis SA, Lausanne (CH)

(72) Inventors: Davide D'Amico, Renens (CH); Anurag Singh, Lausanne (CH); Christopher L. Rinsch, Lausanne (CH)

(21) Appl. No.: 18/872,108

(22) PCT Filed: Jun. 5, 2023
(86) PCT No.: PCT/IB23/00328

§ 371 Date: Dec. 5, 2024

Related U.S. Application Data
(60) Provisional application No. 63/349,327, filed on Jun. 6, 2022.

(51) Int. Cl. A61K 31/37; A61K 31/07; A61K 31/122; A61K 31/355; A61K 31/375; A61K 31/502; A61K 31/616; A61K 31/70; A61K 31/7048; A61K 31/716; A61K 33/00; A61K 35/60; A61K 36/062; A61K 36/21; A61K 36/899; A61K 45/06; A61P 9/04; A61P 9/10; A61P 21/00

(52) U.S. Cl. CPC A61K 31/37; A61K 31/07; A61K 31/122; A61K 31/355; A61K 31/375; A61K 31/502; A61K 31/616; A61K 31/70; A61K 31/7048; A61K 31/716; A61K 33/00; A61K 35/60; A61K 36/062; A61K 36/21; A61K 36/899; A61K 45/06; A61P 9/04; A61P 9/10; A61P 21/00

(57) ABSTRACT

Disclosed are methods and compositions for use in improving cardiac function and health. The methods and compositions are based on urolithins.

### RELATED APPLICATIONS

This application claims the benefit of priority to U.S. Provisional Patent Application No. 63/349,327, filed Jun. 6, 2022.

### BACKGROUND OF THE INVENTION

The rapidly growing and aging population today is confronted with heart dysfunction, such as myocardial infarction, coronary artery disease (CAD), congestive heart failure (CHF), angina, stroke, arrhythmia, fibrillation, peripheral arterial disease (PAD), cardiac disorder, arterial disorder, and post-heart transplantation disorders. These cardiac diseases and disorders require “polymedication” to allow afflicted individuals, particularly middle-aged and elderly individuals, to continue to function optimally and have an improved quality of life with reduced symptomatology related to the heart dysfunction. However, many commonly used pharmaceutical medications prescribed and used to improve or treat cardiac dysfunction can cause or are associated with side effects. There remains an unmet need to improve or treat individuals afflicted with heart dysfunction and reduce side effects of medication.

Urolithins are ellagitannin- and ellagic acid-derived metabolites produced, e.g., by mammalian colonic microflora, including human colonic microflora. Urolithins have potent effects on the improvement of a number of health conditions, and they have been shown to be highly biologically active in vitro and in vivo. Urolithins have been proposed as treatments of a variety of conditions including diseases and conditions relating to cardiac dysfunction.

### SUMMARY OF THE INVENTION

One aspect of the invention is methods useful for improving and/or protecting cardiac function, decreasing the rate of decline in cardiac function, preventing decline in cardiac function, managing or treating peripheral arterial disease (PAD).

Accordingly, provided herein are methods of improving cardiac function, comprising administering to a mammal in need thereof an effective amount of a urolithin; wherein the effective amount of the urolithin is about 10 mg/kg to about 30 mg/kg; and the mammal has a myocardial infarction, coronary artery disease (CAD), congestive heart failure (CHF), angina, stroke, arrhythmia, fibrillation, peripheral arterial disease (PAD), or a cardiac or arterial disorder.

In some embodiments, the urolithin is administered after ischemic reperfusion injury (e.g., at least about one day, about two days, about three days, about four days, about five days or about one week after ischemic reperfusion injury).

In certain embodiments, heart hypertrophy induced by myocardial infarction is decreased in the mammal (e.g., heart hypertrophy in the left ventricle is decreased).

In certain aspects, provided herein are methods of improving cardiac function, comprising administering to a mammal in need thereof an effective amount of a urolithin; wherein the effective amount of the urolithin is about 10 mg/kg to about 30 mg/kg.

In certain aspects, provided herein are methods of protecting cardiac function, comprising administering to a mammal in need thereof an effective amount of a urolithin; wherein the effective amount of the urolithin is about 10 mg/kg to about 30 mg/kg.

In certain aspects, provided herein are methods of decreasing the rate of decline in cardiac function, comprising administering to a mammal in need thereof an effective amount of a urolithin; wherein the effective amount of the urolithin is about 10 mg/kg to about 30 mg/kg.

In certain aspects, provided herein are methods of preventing decline in cardiac function, comprising administering to a mammal in need thereof an effective amount of a urolithin; wherein the effective amount of the urolithin is about 10 mg/kg to about 30 mg/kg.

In certain aspects, provided herein are methods of managing or treating peripheral arterial disease (PAD), comprising administering to a mammal in need thereof an effective amount of a urolithin; wherein the effective amount of the urolithin is about 10 mg/kg to about 30 mg/kg.

In certain aspects, provided herein are methods of managing or improving tissue oxygenation, comprising administering to a mammal in need thereof an effective amount of a urolithin; wherein the effective amount of the urolithin is about 10 mg/kg to about 30 mg/kg.

In certain aspects, provided herein are methods of improving a cardiac output (e.g., heart rate, stroke volume, heart rate variability) comprising administering to a mammal in need thereof an effective amount of a urolithin; wherein the effective amount of the urolithin is about 10 mg/kg to about 30 mg/kg.

In some embodiments, the cardiac output is heart rate.

In certain aspects, provided herein are methods of increasing ventricular ejection fraction, comprising administering to a mammal in need thereof an effective amount of a urolithin; wherein the effective amount of the urolithin is about 10 mg/kg to about 30 mg/kg.

In some embodiments, left ventricular ejection fraction (LVEF) of the mammal is increased.

In certain embodiments, L VEF of the mammal is about less than 60% about less than 55%, about less than 50%, about less than 45%, about less than 40%, about less than 35%, about less than 30%, or about less than 25%, about less than 20% prior to administration of the urolithin.

In some embodiments, LVEF of the mammal is about 5-75% (e.g., about 15-50%, about 15-40%, about 15-35%, about 20-45%, about 40-49%, or about 41-49%) prior to administration of the urolithin.

In certain embodiments, right ventricular ejection fraction (RVEF) of the mammal is increased.

In certain embodiments, RVEF of the mammal is about less than 60% about less than 55%, about less than 50%, about less than 45%, about less than 40%, about less than 35%, about less than 30%, or about less than 25%, about less than 20% prior to administration of the urolithin.

In some embodiments, RVEF of the mammal is about 5-75% (e.g., about 15-50%, about 15-40%, about 15-35%, about 20-45%, about 40-49%, or about 41-49%) prior to administration of the urolithin.

In certain embodiments, heart function is improved. For example, heart function with preserved ejection fraction (HFpEF) is improved, or heart function with reduced ejection fraction (HFrEF) is improved.

In some embodiments, heart systolic function is improved.

In certain aspects, provided herein are methods of increasing heart systolic function, comprising administering to a mammal in need thereof an effective amount of a urolithin; wherein the effective amount of the urolithin is about 10 mg/kg to about 30 mg/kg.

In some embodiments, heart systolic function of the mammal is increased by about 20-90% (e.g., by about 30-80%, or 50-70%).

In certain embodiments, heart systolic function of the mammal is increased by about 56%.

In some embodiments, heart systolic function of the mammal is increased by about 64%.

In certain embodiments, heart diastolic dysfunction of the mammal is decreased.

In some embodiments, heart diastolic dysfunction of the mammal is decreased by about 5-10%.

In certain aspects, provided herein are methods of increasing heart systolic function and skeletal muscle function, comprising administering to a mammal in need thereof an effective amount of a urolithin; wherein the effective amount of the urolithin is about 10 mg/kg to about 30 mg/kg; and the mammal is an aging mammal.

In some embodiments, heart systolic function of the mammal is increased by at least about 20%, 25%, 30%, 35%, 40%, 45%, 50%, 55% or 60%.

In certain embodiments, skeletal muscle function (e.g., skeletal muscle force) of the mammal is increased by about 1-50% (e.g., by about 2-5%).

In certain aspects, provided herein are methods of maintaining, supporting, or improving blood circulation, comprising administering to a mammal in need thereof an effective amount of a urolithin; wherein the effective amount of the urolithin is about 10 mg/kg to about 30 mg/kg.

In certain aspects, provided herein are methods of maintaining, supporting, or improving heart function, comprising administering to a mammal in need thereof an effective amount of a urolithin; wherein the effective amount of the urolithin is about 10 mg/kg to about 30 mg/kg.

In certain aspects, provided herein are methods of maintaining, supporting, or improving heart muscle function, comprising administering to a mammal in need thereof an effective amount of a urolithin; wherein the effective amount of the urolithin is about 10 mg/kg to about 30 mg/kg.

In some embodiments, heart muscular contractility is increased.

In some embodiments, heart fibrosis is decreased.

In some embodiments, heart mitochondrial function is improved.

In some embodiments, heart mitophagy is improved.

In some embodiments, plasma ceramide levels, acylcarnitine levels, brain natriuretic peptide (BNP) levels, creatine kinase (CK) levels, C-reactive protein (CRP) levels, troponin levels, or galectin-2 levels are decreased.

In some embodiments, heart rate is improved.

In some embodiments, heart rate variability is increased.

In some embodiments, circulation (e.g., blood flow) is improved.

In certain embodiments, the method further comprises transplanting mitochondria to the mammal.

In some embodiments, a mitochondrial injury is improved.

In some embodiments, wherein the urolithin is administered orally.

In some embodiments, the urolithin is administered in the form of a tablet or capsule.

In some embodiments, the urolithin is administered over a period of at least about 1 month (e.g., at least about 2 months, at least about 3 months, at least about 4 months, at least about 5 months, or at least about 6 months)

In some embodiments, the mammal is a human.

In some embodiments, the human is elderly.

In some embodiments, the human is at least about 40 years old (e.g., at least about 50 years old, at least about 60 years old, at least about 70 years old, at least about 80 years old, or at least about 90 years old).

In some embodiments, the effective amount of the urolithin is about 25 mg/kg.

In some embodiments, the urolithin is selected from the group consisting of urolithin A, urolithin B, urolithin C, urolithin D, and any combination thereof.

In some embodiments, the urolithin is selected from the group consisting of urolithin A, urolithin B, and a combination of urolithin A and urolithin B.

In certain embodiments, the urolithin is urolithin A.

In certain embodiments, the urolithin is urolithin B.

In certain embodiments, the urolithin is urolithin C.

In certain embodiments, the urolithin is urolithin D.

In some embodiments, the methods described herein further comprise conjointly administering an additional agent to the mammal.

In certain embodiments, the additional agent is a therapeutic agent (e.g., angiotensin-converting enzyme (ACE) inhibitors, angiotensin receptor-neprilysin inhibitors (ARNi), beta-blockers, mineralocorticoid receptor antagonists (MRAs), sodium-glucose co-transporter 2 (SGLT2) inhibitors (e.g., dapagliflozin, empagliflozin), angiotensin-2 receptor blockers (ARBs), diuretics, If channel blockers, aldosterone antagonists, aspirin, P2Y12 inhibitors, calcium channel blockers, cholesterol-lowering drugs (e.g., statins), antiarrhythmic drugs, hydralazine with nitrate, digoxin, and anticoagulants.

In some embodiments, the additional agent is a nutritional supplement (e.g., omega-3 fatty acids, nitrate-rich beet root, nitric oxide, red yeast rice, beta-glucans, vitamin A, vitamin C, vitamin E, vitamin K, potassium salts, magnesium salts, calcium salts, iron salts, manganese salts, copper salts, zinc salts, and phosphate salts).

### BRIEF DESCRIPTION OF THE DRAWINGS

FIG. 1 is a schematic depicting the study design in Example 1.

FIG. 2A shows impact of Urolithin A (UA) on cardiac systolic function measured as Ejection Fraction and Fractional Shortening in mice subjected to Myocardial Infarction (MI). Sham animal used as controls. *p<0.05 unpaired Student t-test.

FIG. 2B shows impact of Urolithin A (UA) on cardiac diastolic function measured as Isovolumetric Relaxation Time in mice subjected to Myocardial Infarction (MI). Sham animal used as controls. *p<0.05 unpaired Student t-test.

FIG. 2C shows impact of Urolithin A (UA) on cardiac hypertrophy measured as Isovolumetric Relaxation Time in mice subjected to Myocardial Infarction (MI). Sham animal used as controls. *p<0.05 unpaired Student t-test.

FIG. 3 shows a gene set analysis. Several gene sets that are at the same time repressed with the occurrence of MI (MIvs sham, grey bars) and also induced by Urolithin A in diseased animals (MI+UA, red bars).

FIG. 4 is a schematic depicting study design in Example 2.

FIG. 5A shows Urolithin A increases fractional shortening in old mice both basally (+32%) and after high-workload (+39%). *p<0.05 Left: Two-way ANOVA (treatment effect).

FIG. 5B shows Urolithin A significantly prevents age-related decline in skeletal muscle function. Unpaired Student t-test.

FIG. 6A shows the impact of Urolithin A on mitochondrial area, expressed as mm2.

FIG. 6B shows images of cristae ultrastructure.

FIG. 7 shows images of cristae ultrastructure of the heart mitochondria and the impact of Urolithin A on Cristae volume density and Cristae number. Urolithin A administration significantly increases cristae volume density and number in old mice.

FIG. 8 shows the impact of Urolithin A on the number of mitolysosomes.

FIG. 9 shows data regarding several gene sets that are at the same time repressed with aging and also induced by Urolithin A in old animals. Normalized enrichment score indicates downregulation of a gene set when value is negative, and upregulation of a gene set when value is positive.

FIG. 10 shows enzymatic activity of the mitochondrial complex II enzyme SDH establishing that administration of UA to old mice significantly increases SHD activity in the heart compared to old vehicle control mice.

### DETAILED DESCRIPTION OF THE INVENTION

The development of methods for improving cardiac function and health with urolithins required substantial innovation. Quantitative analysis of heart systolic function, diastolic function, hypertrophy, and skeletal muscle function was performed to demonstrate Urolithin A has a protective effect against heart failure and a cardioprotective effect during natural aging.

### Definitions

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

“Optional” or “optionally” means that the subsequently described event or circumstances may or may not occur, and that the description includes instances where said event or circumstance occurs and instances in which it does not. For example, “optionally substituted aryl” means that the aryl radical may or may not be substituted, and that the description includes both substituted aryl radicals and aryl radicals having no substitution.

As used herein, the term “treat” as used in connection with a disease, disorder, or condition of a subject, means to reduce by a detectable amount at least one clinical or objective manifestation of the disease, disorder, or condition of a subject. In one embodiment, the term “treat” used in connection with a disease, disorder, or condition of a subject, means to cure the disease, disorder, or condition of a subject.

As used herein, a “food product” refers to a product prepared from a natural food. Non-limiting examples of food products include juices, wines, concentrates, jams, jellies, preserves, pastes, and extracts.

As used herein, a “nutritional supplement” refers to a product suitable for consumption or other administration principally for its health-promoting properties rather than its caloric content.

As used herein, the term “alkyl” refers to a straight chain or branched, noncyclic or cyclic, unsaturated or saturated aliphatic hydrocarbon radical containing carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

As used herein, the term “aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

As used herein, the term “monosaccharide” refers to a simple sugar of the formula (CH2O)n. The monosaccharides can be straight-chain or ring systems, and can include a saccharose unit of the formula —CH(OH)—C(═O)—. Examples of monosaccharides include erythrose, threose, ribose, arabinose, xylose, lyxose, allose, altrose, glucose, mannose, gulose, idose, galactose, talose, erythulose, ribulose, xyulose, psicose, fructose, sorbose, tagatose, erythropentulose, threopentulose, glycerotetrulose, glucopyranose, fructofuranose. In certain embodiments, monosaccharide refers to glucopyranose.

As used herein, the term “oligosaccharide” refers to saccharide consisting of at least two, up to 10 glycosidically linked monosaccharide units, preferably of 2 to 8 monosaccharide units, more preferably of 2 to 7 monosaccharide units, and even more preferably of 2 to 6 monosaccharide units or of 2 to 5 monosaccharide units.

The term “substituted” as used herein (for example, in the context of a substituted heterocyclyl or substituted aryl) means that at least one hydrogen atom is replaced with a substituent. “Substituents” within the context of this invention include halogen, hydroxy, oxo, cyano, nitro, imino, thioxo, amino, alkylamino, dialkylamino, alkyl, alkoxy, alkylthio, haloalkyl, aryl, aralkyl, heteroaryl, heteroarylalkyl, heterocycle and heterocyclealkyl, as well as —NRaRb, —NRaC(═O)Rb, —NRaC(═O)NRaNRb, —NRaC(═O)ORb-NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra, —S(═O)2ORa, ═NSO2Ra and —SO2NRaRb. In the foregoing, Ra and Rb in this context may be the same or different and independently hydrogen, alkyl, haloalkyl, cycloalkyl, aryl, aralkyl, heterocyclyl. In addition, the foregoing substituents may be further substituted with one or more of the above substituents.

### Methods of the Invention

In accordance with the instant invention, it has now been discovered that, urolithins, unexpectedly improve heart systolic function, diastolic function, hypertrophy, and skeletal muscle function. The methods of the invention are useful for improving and/or protecting cardiac function, decreasing the rate of decline in cardiac function, preventing decline in cardiac function, managing or treating peripheral arterial disease (PAD).

In certain embodiments in accordance with this and other aspects of the invention, the urolithin is urolithin A. In certain embodiments in accordance with this and other aspects of the invention, the urolithin is urolithin B. In certain embodiments in accordance with this and other aspects of the invention, the urolithin is urolithin C. In certain embodiments in accordance with this and other aspects of the invention, the urolithin is urolithin D.

In certain aspects, provided herein are methods of improving cardiac function, comprising administering to a mammal in need thereof an effective amount of a urolithin; wherein the effective amount of the urolithin is about 10 mg/kg to about 30 mg/kg; and the mammal has a myocardial infarction, coronary artery disease (CAD), congestive heart failure (CHF), angina, stroke, arrhythmia, fibrillation, peripheral arterial disease (PAD), or a cardiac or arterial disorder.

Ischemia/reperfusion (I/R) injury is a causal factor contributing to morbidity and mortality. The vulnerability of the liver to I/R injury is a major obstacle to liver resection and transplantation surgery where reperfusion after sustained ischemia is unavoidable during hepatectomy and vascular reconstruction. Mitochondrial dysfunction is known to be one of the critical downstream events that lead to I/R-mediated cell death.

In some embodiments, the urolithin is administered after ischemic reperfusion injury (e.g., at least about one day, about two days, about three days, about four days, about five days or about one week after ischemic reperfusion injury).

In certain embodiments, heart hypertrophy induced by myocardial infarction is decreased in the mammal (e.g., heart hypertrophy in the left ventricle is decreased).

In certain aspects, provided herein are methods of improving cardiac function, comprising administering to a mammal in need thereof an effective amount of a urolithin; wherein the effective amount of the urolithin is about 10 mg/kg to about 30 mg/kg.

In certain aspects, provided herein are methods of protecting cardiac function, comprising administering to a mammal in need thereof an effective amount of a urolithin; wherein the effective amount of the urolithin is about 10 mg/kg to about 30 mg/kg.

In certain aspects, provided herein are methods of decreasing the rate of decline in cardiac function, comprising administering to a mammal in need thereof an effective amount of a urolithin; wherein the effective amount of the urolithin is about 10 mg/kg to about 30 mg/kg.

In certain aspects, provided herein are methods of preventing decline in cardiac function, comprising administering to a mammal in need thereof an effective amount of a urolithin; wherein the effective amount of the urolithin is about 10 mg/kg to about 30 mg/kg.

In certain aspects, provided herein are methods of managing or treating peripheral arterial disease (PAD), comprising administering to a mammal in need thereof an effective amount of a urolithin; wherein the effective amount of the urolithin is about 10 mg/kg to about 30 mg/kg.

In certain aspects, provided herein are methods of managing or improving tissue oxygenation, comprising administering to a mammal in need thereof an effective amount of a urolithin; wherein the effective amount of the urolithin is about 10 mg/kg to about 30 mg/kg.

In certain aspects, provided herein are methods of improving a cardiac output (e.g., heart rate, stroke volume, heart rate variability) comprising administering to a mammal in need thereof an effective amount of a urolithin; wherein the effective amount of the urolithin is about 10 mg/kg to about 30 mg/kg.

In some embodiments, the cardiac output is heart rate.

In certain aspects, provided herein are methods of increasing ventricular ejection fraction, comprising administering to a mammal in need thereof an effective amount of a urolithin; wherein the effective amount of the urolithin is about 10 mg/kg to about 30 mg/kg.

In some embodiments, left ventricular ejection fraction (LVEF) of the mammal is increased.

In certain embodiments, L VEF of the mammal is about less than 60% about less than 55%, about less than 50%, about less than 45%, about less than 40%, about less than 35%, about less than 30%, or about less than 25%, about less than 20% prior to administration of the urolithin.

In some embodiments, LVEF of the mammal is about 5-75% (e.g., about 15-50%, about 15-40%, about 15-35%, about 20-45%, about 40-49%, or about 41-49%) prior to administration of the urolithin.

In certain embodiments, right ventricular ejection fraction (RVEF) of the mammal is increased.

In certain embodiments, RVEF of the mammal is about less than 60% about less than 55%, about less than 50%, about less than 45%, about less than 40%, about less than 35%, about less than 30%, or about less than 25%, about less than 20% prior to administration of the urolithin.

In some embodiments, RVEF of the mammal is about 5-75% (e.g., about 15-50%, about 15-40%, about 15-35%, about 20-45%, about 40-49%, or about 41-49%) prior to administration of the urolithin.

In certain embodiments, heart function is improved. For example, heart function with preserved ejection fraction (HFpEF) is improved, or heart function with reduced ejection fraction (HFrEF) is improved.

In some embodiments, heart systolic function is improved.

In certain aspects, provided herein are methods of increasing heart systolic function, comprising administering to a mammal in need thereof an effective amount of a urolithin; wherein the effective amount of the urolithin is about 10 mg/kg to about 30 mg/kg.

In some embodiments, heart systolic function of the mammal is increased by about 20-90% (e.g., by about 30-80%, or 50-70%).

In certain embodiments, heart systolic function of the mammal is increased by about 56%.

In some embodiments, heart systolic function of the mammal is increased by about 64%.

In certain embodiments, heart diastolic dysfunction of the mammal is decreased.

In some embodiments, heart diastolic dysfunction of the mammal is decreased by about 5-10%.

Cardiac muscle undergoes progressive decline in mitochondrial function, similar to that observed in skeletal muscle, resulting in an increase in reactive oxygen species, as well as an increase in the accumulation of defective organelles. The clearance of these damaged organelles by autophagy is important for the maintenance of cardiac muscle function. As autophagy decreases with age, promoting autophagy can serve to protect cardiac muscle function.

Cardiac muscle is also strongly exposed to ischemic episodes during cardiac infarcts. The level of cardiac muscle damage that these ischemic episodes produce is strongly dependent on the ability of the cells to mount an effective autophagy response to clear damaged organelles. In aged animals, a defective autophagy response leads to an increase in cardiac muscle damage after ischemic events. Thus, promotion of autophagy during these acute events could serve to protect cardiac muscle from damage.

In certain aspects, provided herein are methods of increasing heart systolic function and skeletal muscle function, comprising administering to a mammal in need thereof an effective amount of a urolithin; wherein the effective amount of the urolithin is about 10 mg/kg to about 30 mg/kg; and the mammal is an aging mammal.

In some embodiments, heart systolic function of the mammal is increased by at least about 20%, 25%, 30%, 35%, 40%, 45%, 50%, 55% or 60%.

In certain embodiments, skeletal muscle function of the mammal is increased by about 1-50% (e.g., by about 2-5%).

In certain aspects, provided herein are methods of maintaining, supporting, or improving blood circulation, comprising administering to a mammal in need thereof an effective amount of a urolithin; wherein the effective amount of the urolithin is about 10 mg/kg to about 30 mg/kg.

In certain aspects, provided herein are methods of maintaining, supporting, or improving heart function, comprising administering to a mammal in need thereof an effective amount of a urolithin; wherein the effective amount of the urolithin is about 10 mg/kg to about 30 mg/kg.

In certain aspects, provided herein are methods of maintaining, supporting, or improving heart muscle function, comprising administering to a mammal in need thereof an effective amount of a urolithin; wherein the effective amount of the urolithin is about 10 mg/kg to about 30 mg/kg.

In some embodiments, heart muscular contractility is increased.

In some embodiments, heart fibrosis is decreased.

In some embodiments, heart mitochondrial function is improved.

In some embodiments, heart mitochondrial quality is improved.

In some embodiments, heart mitochondrial area is increased.

In some embodiments, expression of genes linked to mitochondrial function in the heart tissue is upregulated.

In some embodiments, the genes are genes of the mitochondrial protein-containing complex or genes of respirasomes.

In some embodiments, heart mitophagy is improved.

In some embodiments, plasma ceramide levels, acylcarnitine levels, brain natriuretic peptide (BNP) levels, creatine kinase (CK) levels, C-reactive protein (CRP) levels, troponin levels, or galectin-2 levels are decreased.

In some embodiments, heart rate is improved.

In some embodiments, heart rate variability is increased.

In some embodiments, circulation is improved.

In certain embodiments, the method further comprises transplanting mitochondria to the mammal.

In some embodiments, a mitochondrial injury is improved.

In some embodiments, wherein the urolithin is administered orally.

In some embodiments, the urolithin is administered in the form of a tablet or capsule.

In some embodiments, the urolithin is administered over a period of at least about 1 month (e.g., at least about 2 months, at least about 3 months, at least about 4 months, at least about 5 months, at least about 6 months)

In some embodiments, the mammal is a human.

In some embodiments, the human is elderly.

In some embodiments, the human is at least about 40 years old (e.g., at least about 50 years old, at least about 60 years old, at least about 70 years old, at least about 80 years old, at least about 90 years old).

In some embodiments, the effective amount of the urolithin is about 25 mg/kg.

In some embodiments, the urolithin is selected from the group consisting of urolithin A, urolithin B, urolithin C, urolithin D, and any combination thereof.

In some embodiments, the urolithin is selected from the group consisting of urolithin A, urolithin B, and a combination of urolithin A and urolithin B.

In certain embodiments, the urolithin is urolithin A.

In certain embodiments, the urolithin is urolithin B.

In certain embodiments, the urolithin is urolithin C.

In certain embodiments, the urolithin is urolithin D.

In some embodiments, the methods described herein further comprise conjointly administering an additional agent to the mammal.

In certain embodiments, the additional agent is a therapeutic agent (e.g., angiotensin-converting enzyme (ACE) inhibitors, angiotensin receptor-neprilysin inhibitors (ARNi), beta-blockers, mineralocorticoid receptor antagonists (MRAs), sodium-glucose co-transporter 2 (SGLT2) inhibitors (e.g., dapagliflozin, empagliflozin), angiotensin-2 receptor blockers (ARBs), diuretics, If channel blockers, aldosterone antagonists, aspirin, P2Y12 inhibitors, calcium channel blockers, cholesterol-lowering drugs (e.g., statins), antiarrhythmic drugs, hydralazine with nitrate, digoxin, and anticoagulants.

In some embodiments, the additional agent is a nutritional supplement (e.g., fish oils (e.g., omega-3 fatty acids)), nitrate-rich beet root, nitric oxide, red yeast rice, beta-glucans, vitamin A, vitamin C, vitamin E, vitamin K, potassium salts, magnesium salts, calcium salts, iron salts, manganese salts, copper salts, zinc salts, phosphate salts, coenzyme q10 (Coq10), carnitine, grape seed extract, or cocoa).

### Exemplary Compounds of the Invention

Urolithins can be used to practice any of the methods herein, including but not limited to improving and/or protecting cardiac function, decreasing the rate of decline in cardiac function, preventing decline in cardiac function, managing or treating peripheral arterial disease (PAD).

As used herein, a “urolithin” refers to a compound of Formula I:

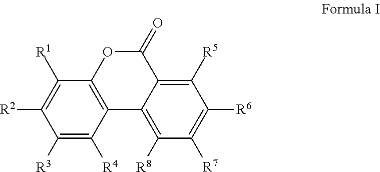

wherein
R¹, R², R³, R⁴, R⁵, R⁶, R⁷, and R⁸ are independently selected from the group consisting of H and OR; and
R is independently for each occurrence H, substituted or unsubstituted alkyl, substituted or unsubstituted aryl, a substituted or unsubstituted monosaccharide, or a substituted or unsubstituted oligosaccharide.

In one embodiment, “urolithin” refers to any one of or a combination of any of urolithin A, urolithin B, urolithin C, and urolithin D.

In some embodiments, the “urolithin” is, or when a combination may include, a glucuronated, methylated, or sulfated urolithin.

In some embodiments, the urolithin is urolithin A.

In some embodiments, the urolithin is urolithin B.

In some embodiments, the urolithin is urolithin C.

In some embodiments, the urolithin is urolithin D.

### Formulations and Clinical Use

The urolithin may be administered, alone or together with another agent, to a subject (e.g., mammal) in a variety of ways. For example, the urolithin can be administered orally or parenterally. Parenterally includes, without limitation, intravenously, intramuscularly, intraperitoneally, subcutaneously, intra-articularly, intrasynovially, intraocularly, intrathecally, topically, or by inhalation. As such, the form of the urolithin dose can be in a variety of forms, including natural foods, processed foods, natural juices, concentrates and extracts, injectable solutions, microcapsules, nano-capsules, liposomes, plasters, inhalation forms, nose sprays, nosedrops, eyedrops, sublingual tablets, and sustained-release preparations.

The compounds of this invention can be provided in isolated form. As used herein, the term “isolated” means substantially removed from other compounds or components with which the compound of interest may otherwise be found, for example, as found in nature. In one embodiment, a compound is isolated when it is essentially completely removed from other compounds or components with which the compound of interest may otherwise be found. In one embodiment, a compound is isolated when it is pure.

The compounds of this invention can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, and intratracheal administration. The active agent may be systemic after administration or may be localized by the use of regional administration, intramural administration, or use of an implant that acts to retain the active dose at the site of implantation.

The compounds of the invention can also be formulated as food additives, food ingredients, functional foods, dietary supplements, medical foods, nutraceuticals, or food supplements.

In pharmaceutical dosage forms, the compounds may be administered in the form of their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier, wherein each dosage unit, for example, mL or L, contains a predetermined amount of the composition containing one or more compounds of the present invention.

Implants for sustained release formulations are well-known in the art. Implants are formulated as microspheres; slabs, etc., with biodegradable or non-biodegradable polymers. For example, polymers of lactic acid and/or glycolic acid form an erodible polymer that is well-tolerated by the host. The implant containing the inhibitory compounds may be placed in proximity to a site of interest, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to, be achieved, and the pharmacodynamics associated with each compound in the host

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

For clinical use, the urolithin is administered in a therapeutically effective amount.

The compounds of the invention can also be formulated as food additives, food ingredients, functional foods, dietary supplements, medical foods, nutraceuticals, or food supplements. In certain embodiments, compounds of the invention can be included into nutraceutical beverages of varying volumes to permit the administration of a daily dose in a convenient format. As a non-limiting example, beverages may deliver effective doses in a final volume ranging from 5 mL to 1,000 mL, delivered as a single dose or multiple doses. In certain embodiments, compositions and methods of the invention are utilized for and in non-human animals. Accordingly, compounds and compositions of the invention may be formulated as veterinary products. Compounds and composition may also be formulated into functional foods for administration to animals, for example, dogs, cats, horses, etc.

Dosing will generally be daily to weekly. In one embodiment, dosing is at least weekly. For example, a subject may receive one dose once weekly, twice weekly, thrice weekly, or every other day. In one embodiment, dosing is at least daily. For example, a subject may receive one or more doses daily.

It is believed that dosing for greatest efficacy in humans involves extended, daily administration. Extended use is contemplated to include use for 1 month, 2 months, 3 months, 4 months, 5 months, 6 months, or even longer.

For clinical use, a urolithin may be administered to treat either a chronic or an acute condition. As autophagy is observed to be increased in cells in less than one day, for example after 8 hours, urolithins may be administered in an acute fashion to treat a condition acutely in need an induction of autophagy. Such instances may include conditions due to reperfusion injuries (for example, organ transplantation), heart attack, stroke, ischemic insult, during surgery (for example, during an angioplasty procedure or replacement of a heart valve), or following a traumatic injury.

For clinical use, a urolithin thereof will generally be administered in an amount equal or equivalent to 0.2-2000 milligram (mg) of urolithin per kilogram (kg) of body weight of the subject per day. In one embodiment, the urolithin is administered in a dose equal or equivalent to 2-2000 mg of urolithin per kg body weight of the subject per day. In one embodiment, the urolithin is administered in a dose equal or equivalent to 20-2000 mg of urolithin per kg body weight of the subject per day. In one embodiment, the urolithin is administered in a dose equal or equivalent to 50-2000 mg of urolithin per kg body weight of the subject per day. In one embodiment, the urolithin is administered in a dose equal or equivalent to 100-2000 mg of urolithin per kg body weight of the subject per day. In one embodiment, the urolithin is administered in a dose equal or equivalent to 200-2000 mg of urolithin per kg body weight of the subject per day.

The formulations of urolithin can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments may be used to optimize the dose and dosing frequency for any particular compound.

In one embodiment, the urolithin is administered at a concentration in the range from about 0.001 microgram/kg to greater than about 500 mg/kg. For example, the concentration may be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed by the present invention.

In one embodiment, the urolithin is administered at a dosage in the range from about 0.2 milligram/kg/day to greater than about 100 mg/kg/day. For example, the dosage may be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

In one embodiment, the urolithin is administered at a dosage in the range from about 0.25 milligram/kg/day to about 25 mg/kg/day. For example, the dosage may be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

In another embodiment, the urolithin is administered in concentrations that range from 0.01 micromolar to greater than or equal to 500 micromolar. For example, the dose may be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed by the present invention.

In yet another embodiment, the urolithin is administered at concentrations that range from 0.10 microgram/mL to 500.0 microgram/mL. For example, the concentration may be 0.10 microgram/mL, 0.50 microgram/mL, microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL. 250.0 microgram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed by the present invention.

Additional non-limiting, illustrative examples of urolithin doses that may be used are provided in Table 1.

TABLE 1
Urolithin Dosing in Mouse and Human

| Mouse Dose (mg/kg/day) | Human Dose (mg/kg/day) |
|---|---|
| 5 | 0.36 |
| 10 | 0.73 |
| 15 | 1.09 |
| 20 | 1.46 |
| 30 | 2.19 |
| 40 | 2.91 |
| 50 | 3.64 |
| 60 | 4.37 |
| 70 | 5.10 |
| 80 | 5.33 |
| 90 | 6.56 |
| 100 | 7.29 |
| 110 | 8.01 |
| 120 | 8.74 |
| 130 | 9.47 |
| 140 | 10.20 |
| 150 | 10.93 |
| 160 | 11.66 |
| 170 | 12.38 |
| 180 | 13.11 |
| 190 | 13.84 |
| 200 | 14.57 |
| 210 | 15.30 |
| 220 | 16.03 |
| 230 | 16.76 |
| 240 | 17.48 |
| 250 | 18.21 |
| 260 | 18.94 |
| 270 | 19.67 |
| 280 | 20.40 |
| 290 | 21.13 |
| 300 | 21.86 |
| 310 | 22.58 |
| 320 | 23.31 |
| 330 | 24.04 |
| 340 | 24.77 |
| 350 | 25.50 |
| 360 | 26.23 |
| 370 | 26.96 |
| 380 | 27.68 |
| 390 | 28.41 |
| 400 | 29.14 |
| 410 | 29.87 |
| 420 | 30.60 |
| 430 | 31.33 |

Additional non-limiting, illustrative examples of urolithin dose ranges that may be used are provided in Table 2.

TABLE 2
Urolithin Dosing in Humans
Human Dose Ranges (mg/kg/day)

| Low | High |
|---|---|
| 0.2 | 4 |
| 0.2 | 5 |
| 0.2 | 6 |
| 0.2 | 7 |
| 0.2 | 8 |
| 0.2 | 9 |
| 0.2 | 10 |
| 0.2 | 15 |
| 0.2 | 20 |
| 0.2 | 25 |
| 0.2 | 30 |
| 0.2 | 35 |
| 0.2 | 40 |
| 0.5 | 2 |
| 0.5 | 3 |
| 0.5 | 4 |
| 0.5 | 5 |
| 0.5 | 10 |
| 0.5 | 15 |
| 0.5 | 20 |
| 0.5 | 25 |
| 0.5 | 30 |
| 0.5 | 35 |
| 0.5 | 40 |
| 0.75 | 5 |
| 0.75 | 10 |
| 0.75 | 15 |
| 0.75 | 20 |
| 0.75 | 25 |
| 0.75 | 30 |
| 0.75 | 35 |
| 0.75 | 40 |
| 1 | 2 |
| 1 | 3 |
| 1 | 4 |
| 1 | 5 |
| 1 | 6 |
| 1 | 7 |
| 1 | 8 |
| 1 | 9 |
| 1 | 10 |
| 1 | 11 |
| 1 | 12 |
| 1.25 | 4 |
| 1.25 | 5 |

Any dose may be given as a single dose or as divided doses.

In one embodiment, the urolithin is administered in a dose sufficient to achieve a peak serum level of urolithin and its known metabolites (glucoronides, sulfonates, etc.) of at least 0.001 micromolar (μM). In one embodiment, the urolithin is administered in a dose sufficient to achieve a peak serum level of urolithin of at least 0.01 μM. In one embodiment, the urolithin is administered in a dose sufficient to achieve a peak serum level of urolithin of at least 0.1 μM. In one embodiment, the urolithin is administered in a dose sufficient to achieve a peak serum level of urolithin of at least 1 μM. In various embodiments, the urolithin is administered in a dose sufficient to achieve a peak serum level of urolithin of at least 10 μM, at least 20 μM, at least 30 μM, at least 40 μM, at least 50 μM, at least 60 μM, at least 70 μM, at least 80 μM, at least 90 μM, at least 100 μM, or at least 200 μM.

In one embodiment, the urolithin is administered in a dose sufficient to achieve a sustained serum level of urolithin of at least 0.001 micromolar (μM). In one embodiment, the urolithin is administered in a dose sufficient to achieve a sustained serum level of urolithin of at least 0.01 μM. In one embodiment, the urolithin is administered in a dose sufficient to achieve a sustained serum level of urolithin of at least 0.1 μM. In one embodiment, the urolithin is administered in a dose sufficient to achieve a sustained serum level of urolithin of at least 1 μM. In one embodiment, the urolithin is administered in a dose sufficient to achieve a sustained serum level of urolithin of at least 10 μM, the urolithin is administered in a dose sufficient to achieve a sustained serum level of urolithin of at least 50 μM. The sustained serum level can be measured using any suitable method, for example, high pressure liquid chromatography (HPLC) or HPLC-MS.

### Examples

The invention now being generally described, it will be more readily understood by reference to the following, which is included merely for purposes of illustration of certain aspects and embodiments of the present invention, and is not intended to limit the invention.

### Example 1: Urolithin a Administration after Myocardial Infraction

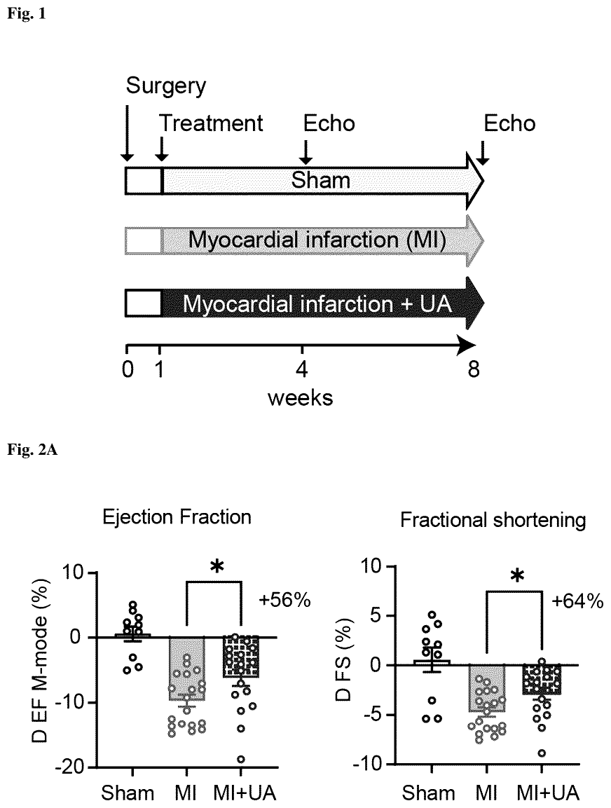

The goal of the study is to assess urolithin A treatment effect on heart function after myocardial infarction. FIG. 1 shows a schematic of the study design, and Table 3 shows the experimental design. Myocardial infarction (MI) was induced in 6-week old Wistar male rats from groups 1, 2, and 3 (Table 3), by permanent occlusion of Left Anterior Descending coronary artery (ligation was performed at Do).

TABLE 3

| Group | N/group | Disease induction | Treatment | Cardiac assessment |
|---|---|---|---|---|
| 1 | 10 | No induction - Surgery | Daily PO administration | Echocardiography |
| | | without LAD ligation (Sham) | for 2M-Vehicle | Heart Mass |
| 2 | 20 | LAD ligation | Daily PO administration | Echocardiography |
| | | | for 2M-Vehicle | Heart Mass |
| 3 | 20 | LAD ligation | Daily PO administration | Echocardiography |
| | | | for 2M-Urolithin A | Heart Mass |

Rats were anesthetized with an intraperitoneal (IP) injection of Medetomidine (0.5 mg/kg) and ketamine (50 mg/kg), intubated and ventilated at 10 mL/kg tidal volume and 70-80 cycles/min. Body temperature were maintained between 36.5° C. and 37.5° C. via a thermoregulated heating pad connected to a rectal probe. Rats were placed in supine position, chest-shaved and prepared for standard surgical conditions. A left lateral thoracotomy was carefully performed so as to expose the heart. Then, a suture was placed around the LAD (4/0 Silk, Ethicon), around 2 mm below the left atrium and close to the interventricular junction, in order to obtain infarct size (IS) near 40% of the total left ventricular (LV) area. Chest was closed, air was expelled from the rib cage to avoid pneumothorax and quick reanimation was performed using atipamezole hydrochloride (IM, 0.5 mg/ml, Zoetis).

A peri-operative care of the animals was performed during the surgery:
Pre-operative care by one carprofen injection (SC, 5 mg/kg, Pfizer) and one lidocaine injection on the site of incision (SC, 4 mg/kg, Vetoquinol).
Post-operative care: 3 injections of buprenorphine (SC, 0.01 mg/kg, Axience) will be administered after awakening and during the first 24 hours after the surgery.
   SHAM (group 1)

Animals from group 1 were subjected to the same protocol as MI groups. However, after the left lateral thoracotomy exposing the heart, the rib cage were closed without passing the suture thread around LAD.

Urolithin A or vehicle were administered by gavage at 25 mg/kg starting one week after myocardial infraction. Heart and plasma samples were collected at each time point. Ejection Fraction (EF), Fractional Shortening (FS), Isovolumic relaxation time (IVRT) were measured by echocardiography at 1 month and 2 month time points to evaluate heart function.

### Echocardiography

Rats underwent transthoracic echocardiographic examination in order to assess cardiac morphology and function in a noninvasively way. Echocardiography was performed by using a digital ultrasound system (Vivid 7, GE Medical Systems) equipped with a 10 MHz phased-array and a 13 MHz linear-array transducer. All images acquisitions, measurements and calculations were done in accordance to the American Society of Echocardiography (Schiller et al., J Am Soc Echocardiogr 1989) and previous validation of the method in rat (Litwin et al., Circulation 1994).

Rats were anesthetized with 4% isoflurane in 50% oxygen-50% air mixture and maintained with 2% isoflurane during the procedure. They were placed in supine position on a heating pad and thorax-shaved. Body temperature was monitored and maintained between 36.5-37.5° C. via a rectal probe connected to the thermoregulated heating pad.

Standard B-mode (Brightness-mode) and M-mode (Motion-mode) images of the heart were obtained in the two-dimensional (2D) parasternal long axis view (PSLA). LV parameters will be measured and calculated as the mean of 3 consecutive cardiac cycles by a single blinded trained operator.
Left Ventricular function
   LV function is calculated using the LV dimensions parameters previously described.
FS=fractional shortening=[(LVID_{d}−LVIDₛ)/LVID_{d}]*100. It is a calculation based on the difference of dimensions between systole and diastole measured in M-mode. It is a reflection of LV contractility
EF=ejection fraction=[(End diastolic volume−End systolic volume)/End diastolic volume]*100. It is a calculation based on the difference of volumes between systole and diastole measured in B-mode and M-mode. It is a reflection of LV contractility.
   Additional echocardiographic measurement on rats included analysis of IVRT, which is the period from aortic valve closure to mitral valve opening and is an indicator of diastolic dysfunction. A total of four (3) echocardiographic exams were performed for all the animals.

The first examination will take place 5 days after surgery (Table 4). It will be used as a control of the surgery to exclude rats with small infarcts and limited reduction of EF. Two exclusion criteria will be used:
IVS not akinetic (infarct too small)
EF>45%

Table 4 represents a theoretical schedule for the two first weeks of experimentation with days dedicated to surgeries and those dedicated to the first echocardiography 5 days after the surgery.

TABLE 4
Theoretical schedule

| | Monday | Tuesday | Wednesday | Thursday | Friday | Saturday | Sunday | Monday | Tuesday | Wednesday | Thursday | Friday |
|---|---|---|---|---|---|---|---|---|---|---|---|---|
| A.M. | | | Surgery | Surgery | Surgery | | | | | Surgery | Surgery | Surgery |
| | | | D 1 | D 2 | D 3 | | | | | D 4 | D 5 | D 6 |
| P.M. | | | | | | | | Echo D 1 | Echo D 2 | Echo D 3 | | |

D, day;
Echo, echocardiography

MI rats included (groups 2 and 3) were randomized in three homogeneous groups based on the following parameters:
LVID_{d}
EF
Body weight

Sham animals were present the same parameters of LV dimension and function, due to the absence of LAD ligation, but should present the same body weight. Randomization was realized by session, after the first echocardiography. After the randomization, a longitudinal follow-up study by echocardiography was performed on the 4 groups of rats.

The 2^{nd} and 3^{rd} echocardiographic examinations were done at Month Two (M2) following the start of the diets. They were used to assess the cardiac remodeling following MI, based on the echocardiographic parameters previously described.

Due to the number of rats planned for the study, at least three sessions of surgery were performed on four consecutive weeks. Thus, cardiac evaluation at M2 was only available at the end of the analysis of the three sessions.

TABLE 5
Candidate compounds
Test items

| | Dose (mg/kg)/ | | Route of |
|---|---|---|---|
| Nature | schedule | Vehicle | administration |
| Uroltihin A | 25/Daily from | 0.5% CMC | PO |
| | D₀ to D₆₀ | | |

D, day;
PO, per os;
kg, kilogram;
mg, milligram;

The requested amount of Uroltihin A was defined on the basis of the dose to be tested, the total number of animals receiving the compound, and a mean final body weight around 400 g.

Preparation of candidate compounds: Vehicle: 0.5% carboxymethylcellulose

Preparation: It was prepared one solution per test compound and per concentration using 0.5% carboxymethylcellulose as vehicle on a weekly basis. Each solution was sonicated for 15 min at 37° C. to enable optimal dissolution of compounds.

### Frequency of Preparation: Every Week

Storage: Each formulation was then be aliquoted into 7 tubes, which was stored at −80° C. For compounds that are expected to form suspensions (due to low solubility), formulations were put for stirring using a magnet during the process of aliquoting. One aliquot will be thawed every day and the volume of gavage will be adapted based on the last measured body weight.

### PO Administrations

The PO administrations were performed in animals from groups 1 to 3 (Table 3) in accordance with standard operating procedure in force. The PO administrations were performed in un-anaesthetized animals using an oral gavage probe.

The final volume of administration was set at 5 ml/kg. According to our procedures, the volume of administration were adjusted every week on the basis of the mean body weight of the animals of the corresponding group. If an animal had a weight standing more than 20% compared to the mean body weight of its group, the volume of administration would be adjusted especially for this animal on the basis of its own body weight. On the day of terminal recordings, animals received the volume of administration corresponding to its own body weight. Rats were treated orally with Vehicle and Candidate compounds 1 and 2 once daily, 7 days a week. Treatment started one week after the MI at will last for 2 months.

### Mortality

Surgery (MI) can be associated with mortality during the first 24 hours. The use of extra animals was assumed in order to make sure that 70 animals were included for the experiment. Over time, MI can be associated with mortality, which is model dependent.

After the first echocardiography and randomization, deviation were notified a soon as possible. Dead animals were not replaced after the first echocardiography.

### Euthanasia and Collection of Heart Tissue

At the end of the procedure, after the last echocardiography, animals were deeply anesthetized with 2,5% isoflurane in 50% O₂/50% air mixture following buprenorphine injection (0.03 mg/kg, SC). One blood sample was collected at the end of the procedures through the jugular vein before sacrifice. A volume of at least 0.4 ml of blood was collected on a K3-EDTA-coated tube and was centrifuged for 10 minutes (10000 rpm, 20° C.). 200 microliters of plasma was frozen and stored at approximately −80° C. until shipment to the sponsor.

Then, hearts were arrested in diastole (KCl 2M, 1 ml/kg) for the subsequent histological analysis by jugular injection. The atria were removed and the heart were washed in cold phosphate buffer saline (PBS, 4° C.) and quickly sponged to remove the liquid from the ventricles. Then, the right ventricular (RV) wall was separated from the remaining part of the heart (LV+Septum) and LV+S was carefully weighed, using an analytical balance (AX 205 (Mettler Toledo) or equivalent). Finally, RV was removed and LV+S was collected and processed as followed:
Fixed LV+S: They were put in NBF 10% during 48 hours and were transferred in ethanol 70% before paraffin inclusion. (Group 1, n=10; groups 2 to 4, n=20).

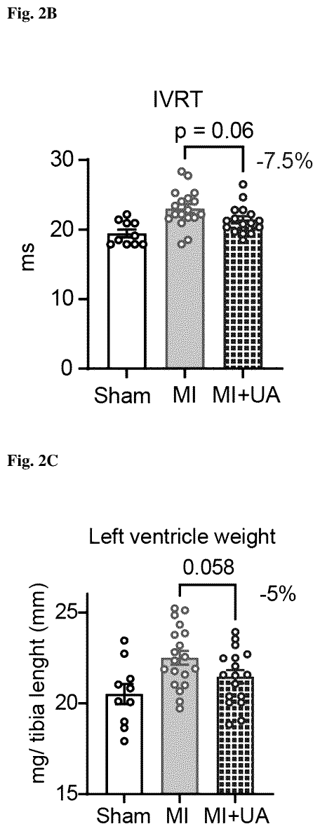

Results of the study are shown in FIGS. 2A-2C. Urolithin A significantly improves systolic dysfunction measured as Ejection fraction and Fractional shortening (FIG. 2A), improves diastolic function, measured as isovolumetric relaxation time (FIG. 2B), and reduces cardiac hypertrophy, calculated as heart weight over tibia length (FIG. 2C). In conclusion, Urolithin A has a protective effect against heart failure.

### Example 1A: Impact of Urolithin a on Gene Sets Associated with Improved Mitochondrial Function

### RNA Extraction and Quality Control

Heart sections from rats in the groups described in FIG. 1 (Example 1) have been prepared using a microtome, deparaffinized and RNA extracted using the following kit (RNAeasy FFPE 76504, Qiagen). Upon extraction, RNA was quality controlled with Agilent Fragment Analyzer System.

### Library Preparation and Sequencing

Library preparation was performed using Strand-specific cDNA library and following the steps here indicated:
rRNA depletion.
mRNA fragmentation.
Random primed cDNA synthesis (strand-specific)
Adapter ligation and adapter specific PCR amplification
   RNA-seq run was executed with the NovaSeq6000 using S4 flowcells with 2×150 bp.

### Bioinformatic Analysis

QC and preprocessing of raw sequencing reads was performed using an in-house QC pipeline. The pipeline utilizes fastp, version 0.23.2 (Chen, A. et al. Bioinformatics 2018, 34(17), i884-i890), that is an all-in-one preprocessing tool for FastQ files, automating the quality control, quality trimming and adapter clipping procedures. High quality RNA-seq read pairs were mapped to the Ensembl rat reference genome

Reads with a minimum PHRED score of 15, a minimum length of 15 bp, and a minimum unqualified percent limit of 40 (40%) were retained for downstream analysis.

Before the DE analysis, a pre-filtering step was applied to remove low abundance mRNA measurements, by excluding genes with less than 10 total counts across samples. Additionally, an independent filtering procedure of DESeq2 was enabled, in order to filter out genes with very low counts that are unlikely to show significant alterations in gene expression. Gene symbols, descriptions, and biotypes were matched to Ensembl GTF ids by using the R package biomaRt. v. 2.46.3 (Durink, S. et al. Nature Protocol 2009, 4, 1184-1191). P-values were corrected for multiple-testing using the Benjamini and Hochberg (BH) method (Benjamini, Y. and Hochberg, Y. Journal of the Royal Statistical Society, Series B, 1995, 57, 289-300). Genes exhibiting a corrected (adjusted) p-value lower than 0.05 and an absolute log 2 Fold Change higher than 1 were considered as Differentially Expressed (differentially expressed genes-DEGs).

Gene Set Enrichment Analysis (GSEA) (Subramanian, et al. Proc Natl Acad Sci USA 2005, 102 (43), 15545-50) was performed for characterizing complex relations of gene expression changes with gene ontologies (cellular components), under different biological conditions. GSEA uses all processed genes and sorted them according log 2 Fold Change. Gene ranking information is transformed to pathway level to assign statistical significance for each analyzed gene set. Each gene set is analysed independently, and the direction of gene regulation (up/down regulation) is inferred by using the examined biological comparison.

### Results

A Gene set enrichment analysis was performed looking at gene set (i.e., biological pathways) belonging to the category “Cellular Components”. In particular, it was investigated as to which biological pathways are: (i) significantly down-regulated when comparing rats with myocardial infarction to healthy control rats (Mi vs sham); (ii) significantly upregulated when comparing MI rats treated with Urolithin A (MI+UA) to rats with MI that we not treated with Urolithin A (MI).

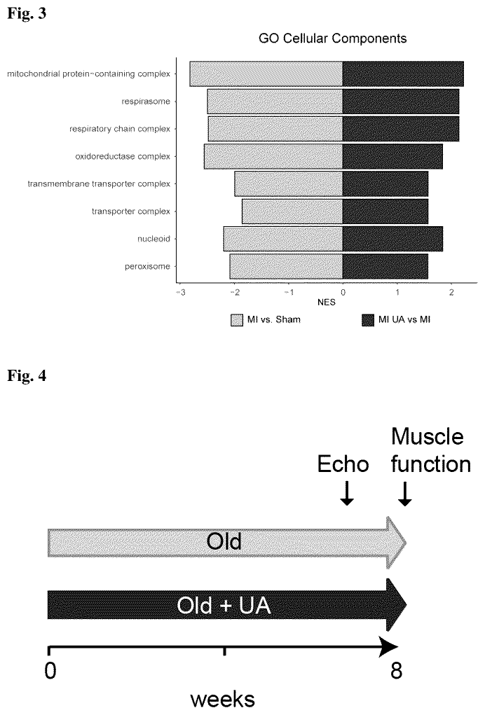

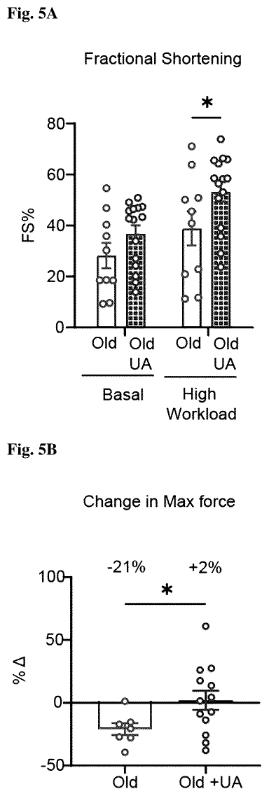

FIG. 3 shows that several gene sets that are at the same time repressed with the occurrence of MI (MI vs sham, grey bars) and also induced by Urolithin A in diseased animals (MI+UA, dark gray bars). The X axis represents the Normalized enrichment score that indicates downregulation of a gene set when value is negative; and upregulation of a gene set when value is positive.

Several of these gene sets are associated with mitochondrial function, such as the mitochondrial protein-containing complex, that contains genes essential for optimal mitochondrial function; and respirasome, that contains genes required for proper mitochondrial respiration (i.e., energy production).

These data indicate that myocardial infarction is associated with reduced expression of genes linked to mitochondrial function in the heart tissue. And that administration of Urolithin A can positively revert the expression of these gene sets.

Additional gene sets, repressed with the occurrence of MI (MI vs sham, grey bars) and also induced by Urolithin A in diseased animals (MI+UA, red bars) are related to biology of nucleoids and peroxisomes.

### Example 2: Preclinical In Vivo Study to Assess Improvement of Both Heart and Skeletal Muscle Function

The goal of the study is to evaluate urolithin treatment effect age-related heart dysfunction. FIG. 3 shows a schematic of study design. Old C57BL/6 mice (26 months) were used as model to reproduce natural decline in heart function with aging. Chow diet with and without urolithin A was administered for 2 months at 50 mg/kg. Post-mortem heart muscles, skeletal muscles, and plasma samples post-mortem were collected at the end of the study. Ejection Fraction (EF), Fractional Shortening (FS) were measured by echocardiography. Muscle contractility was also measured. Analysis of both heart and skeletal muscle function were conducted at the end of the study.

Results are shown in FIGS. 4A-4B. Urolithin A significantly improves both heart and skeletal muscle function during natural aging. FIG. 4A shows Urolithin A increases fractional shortening in old mice both basally and after high-workload, and FIG. 4B shows Urolithin A significantly prevents age-related decline in skeletal muscle function. In conclusion, Urolithin A has a cardioprotective effect during natural aging.

### Example 3: Impact of Urolithin a on Mitochondrial Morphology Associated with Improved Mitochondrial Function

The goal of the study was to evaluate urolithin treatment effect on age-related heart dysfunction. Old C57BL/6 mice (24 months at the end of the study) were used as model to reproduce natural decline in heart function with aging and administered by gavage for 2 months with either Urolithin A at 50 mg/kg (Old UA) or vehicle (Old Vehicle). Young mice (18 weeks-old at the end of the study) were administered with vehicle for 2 months. Post-mortem heart muscles, were collected at the end of the study.

### Transmission Electron Microscopy Methods and Analysis

Muscle samples from animals as described in example 2 were fixed in 2% glutaraldehyde solution 0.1 M Sorenson's buffer (pH 7.3). These samples were later postfixed in 1% osmium tetroxide for 8 min [pulse microwaved (MW), 100 watts (W)] and rinsed in distilled H2O (3×150 W 40 s per step). Samples were dehydrated in a graded series of acetone (25%; 50%; 75%; 3× 100%; 150 W 40 seconds per step) before being impregnated with increasing concentrations of epoxy resin (TAAB medium resin) in acetone (25%; 50%; 75%; 3×100%; 300 W 3 min per step). The samples were then embedded in 100% fresh resin and left to polymerize at 60° C. in a conventional oven for a minimum of 24 h.

All resin blocks were trimmed using a razor blade to form a trapezoid block face. Sections were cut in a longitudinal or transverse orientation on an ultramicrotome (Wolf et al., 2019) using a diamond knife. Semithin sections (0.5 μm) were stained with toluidine blue and viewed on a light microscope (LM) to verify orientation of tissue. Ultrathin sections (70 nm) were then cut and picked up onto copper grids. Sections from blocks were stained with 1% uranyl acetate (30 min) and 3% lead citrate (7 min). All sections were examined using a HT7800 120 kV TEM (Hitachi). Digital micrographs were captured using an EMSIS Xarosa CMOS Camera with Radius software.

These samples were later postfixed in 1% osmium tetroxide during 1 h, dehydrated within an increasing concentration of acetone. To verify the orientation of the muscle tissue, 1 μm thick sections were stained with toluidine blue prior to ultrathin sectioning. Ultrathin sections were cut in a longitudinal or transverse orientation on an Ultracut ultramicrotome (Leica). These sections were then stretched to remove compressions and mounted on copper grids before being stained with 2% aqueous uranyl acetate and lead citrate (Leica). Sections were then imaged using a Philips CM 100 electron microscope. Digital micrographs were captured using an AMT XR80 CCD digital camera at ×7900 magnification.

Individual heart mitochondria from 3 Young vehicle, 3 Old vehicle and 3 Old UA O-AL mice were manually traced in transverse orientations using ImageJ software (NIH) (https://imagej.nih.gov/ij/) and to quantify the following morphological and shape descriptors: area (μm2), perimeter (μm), circularity 4π×(area/perimeter2), aspect ratio ((major axis)/(minor axis)): the aspect ratio being a measure of the length to width ratio, form factor ((perimeter)/(4π×surface)): a measure sensitive to the complexity and the branched appearance of the mitochondria, Feret's diameter (the longest distance (μm) between two points of the mitochondria studied) and minimum Feret diameter (corresponds to the smallest diameter of the mitochondria) (Picard et al., 2012). It is important to highlight here that n=3-4 is a sample size commonly used in TEM-based studies investigating mitochondrial morphology and ultrastructure.

### RNA-Seq

RNA-seq analysis from samples derived from example 3 has been performed as described for example 1.

### Succinate Dehydrogenase Activity

Hearts in OCT were cut into sections of 8 μM using a cryostat. Sections were then incubated in a solution containing nitroblue tetrazolium (1.5 mM), sodium succinate (130 mM), phenazine methosulphate (0.2 mM), and sodium azide (0.1 mM) for 6 min at room temperature. The reaction was stopped by two washes in PBS. Slides were mounted using Fluoromont mounting medium (Thermofisher, 00-4958-02). All samples were processed successively using the same incubation solution ensuring that they underwent the exact same experimental conditions. Images of the tissues were taken with a bright field microscope. The intensity of the staining was analyzed with Image J.

### Results

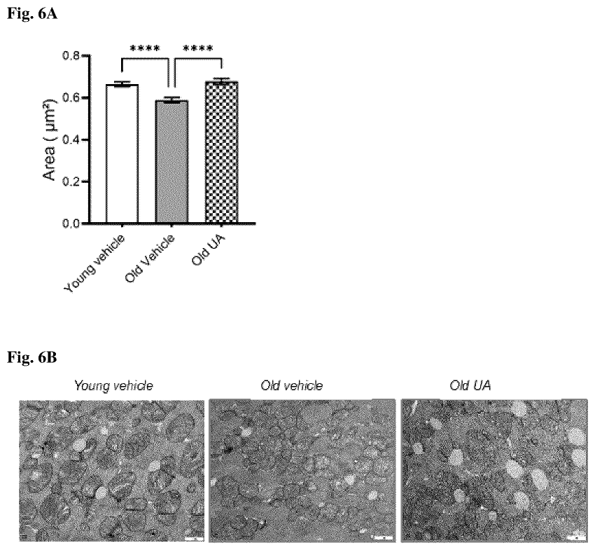

Old mice showed a significant decrease in mitochondrial area, expressed as mm2, compared to young mice. Administration of UA for 2 months in old mice, significantly increased mitochondrial area to levels compared to young animals (FIG. 6A). A decline in mitochondria area has been reported also in the skeletal muscle of elderly human subjects compared to young subjects (Crane, J. D. et al. J Gerontol A Biol Sci Med Sci 2010, 65 (2). 119-128). This decline has been associated with reduced mitochondrial function (Crane, J. D. et al. 2010). This data indicates that UA is able to improve mitochondrial area associated to improved mitochondrial function in the heart with aging.

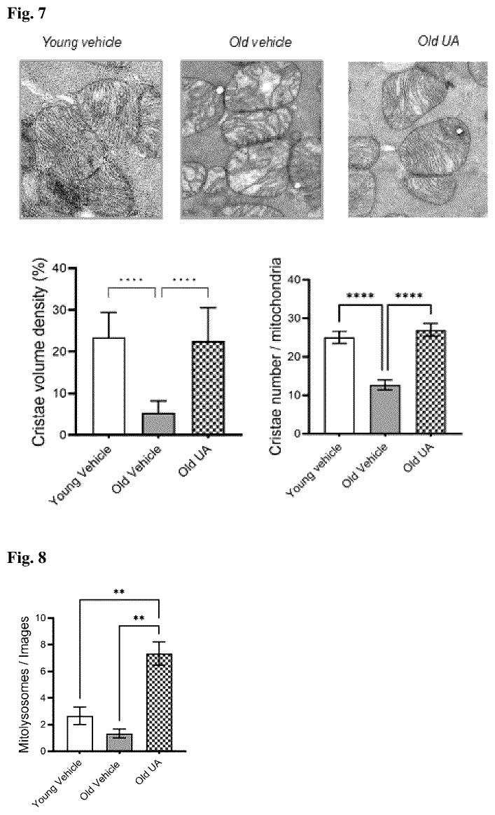

In addition, the cristae ultrastructure of the heart mitochondria was analysed. These represent the complex invaginations of the mitochondrial inner membrane. It is where mitochondrial respiration occurs and therefore maintaining their correct structure is key to preserve optimal mitochondrial bioenergetic capacity (Baker, N. et al. Mitochondrion 2019, 49, 259-268). It was observed that old mice have disrupted cristae structure, quantified as cristae volume and number (FIG. 7), compared to young animals. UA administration significantly increases cristae volume density and number in old mice. This indicates UA ability to improve mitochondrial quality in the heart during aging.

In the heart of the same animals as in this example, mitophagy events we quantified, indicated as the number of mitochondria engulfed into autophagosome vesicles and therefore forming mitolysosomes. It was observed that old mice have reduced mitophagy events, quantified as number of mitolysosomes (FIG. 8), compared to young animals. UA administration significantly increases mitophagy events in old mice. This is clinically relevant as mitophagy was shown to be essential for heart function in experimental models (Kubli, D. A. et al. J Biol Chem 2013, 288 (2), 915-926). Also, mitophagy declines in human patients with heart failure (Billia, F. et al. Proc Natl Acad Sci USA 2011, 108 (23), 9572-7). This indicates UA's ability to increase mitophagy in the heart during aging.

A Gene set enrichment analysis was performed looking at gene set (i.e., biological pathways) belonging to the category “Cellular Components”. In particular, it was investigated as to which biological pathways are: (i) significantly down-regulated when comparing old mice with to young mice; (ii) significantly upregulated when comparing old mice treated with UA to old mice treated with vehicle.

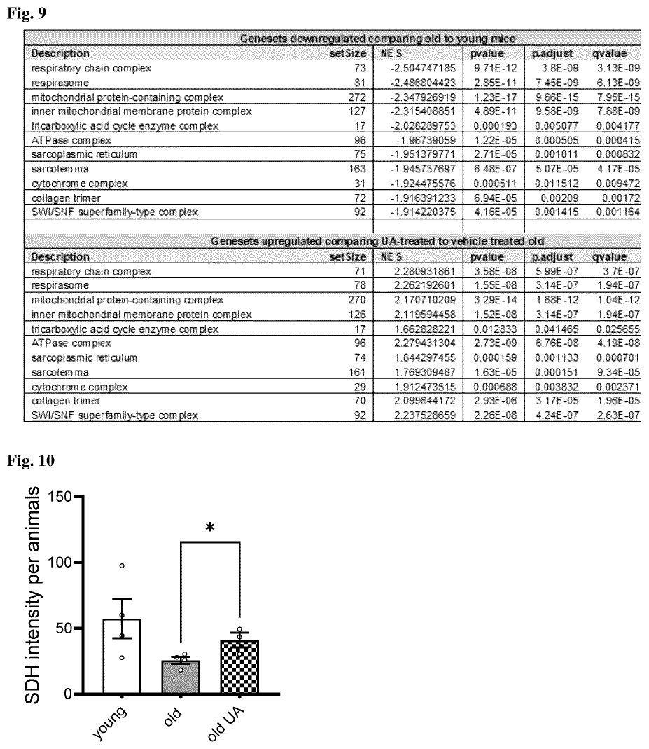

FIG. 9 lists the several gene sets that are at the same time repressed with aging and also induced by Urolithin A in old animals. Normalized enrichment score that indicates downregulation of a gene set when value is negative; and upregulation of a gene set when value is positive.

Several of these gene sets are associated with mitochondrial function, such as respirasome, that contains genes required for proper mitochondrial respiration (i.e., energy production). Additional gene sets are associated to improved heart cellular health by UA treatment, such as sarcolemma.

These data indicate that myocardial infarction is associated with reduced expression of genes linked to mitochondrial function in the heart tissue. And that administration of Urolithin A can positively revert the expression of these gene sets.

To further validate a positive impact of UA on mitochondrial function in the health we measured the enzymatic activity of the mitochondrial complex II enzyme SDH. This immunoenzymatic assay is an indicator of mitochondrial respiratory activity and therefore of mitochondrial function. FIG. 10 shows that UA administration to old mice significantly increases SHD activity in the heart compared to old vehicle control mice.

### INCORPORATION BY REFERENCE

All US patents and US and PCT published patent applications mentioned in the description above are incorporated by reference herein in their entirety.

### EQUIVALENTS

Having now described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

We claim:

1. A method of improving cardiac function, comprising administering to a mammal in need thereof an effective amount of a urolithin; wherein the effective amount of the urolithin is about 10 mg/kg to about 30 mg/kg; and the mammal has a myocardial infarction, coronary artery disease (CAD), congestive heart failure (CHF), angina, stroke, arrhythmia, fibrillation, peripheral arterial disease (PAD), or a cardiac or arterial disorder.

2. The method of claim 1, wherein the urolithin is administered after ischemic reperfusion injury (e.g., at least about one day, about two days, about three days, about four days, about five days or about one week after ischemic reperfusion injury).

3. The method of claim 1 or 2, wherein heart hypertrophy induced by myocardial infarction is decreased in the mammal.

4. The method of claim 3, wherein heart hypertrophy in the left ventricle is decreased.

5. A method of improving cardiac function, comprising administering to a mammal in need thereof an effective amount of a urolithin; wherein the effective amount of the urolithin is about 10 mg/kg to about 30 mg/kg.

6. A method of protecting cardiac function, comprising administering to a mammal in need thereof an effective amount of a urolithin; wherein the effective amount of the urolithin is about 10 mg/kg to about 30 mg/kg.

7. A method of decreasing the rate of decline in cardiac function, comprising administering to a mammal in need thereof an effective amount of a urolithin; wherein the effective amount of the urolithin is about 10 mg/kg to about 30 mg/kg.

8. A method of preventing decline in cardiac function, comprising administering to a mammal in need thereof an effective amount of a urolithin; wherein the effective amount of the urolithin is about 10 mg/kg to about 30 mg/kg.

9. A method of managing or treating peripheral arterial disease (PAD), comprising administering to a mammal in need thereof an effective amount of a urolithin; wherein the effective amount of the urolithin is about 10 mg/kg to about 30 mg/kg.

10. A method of managing or improving tissue oxygenation, comprising administering to a mammal in need thereof an effective amount of a urolithin; wherein the effective amount of the urolithin is about 10 mg/kg to about 30 mg/kg.

11. A method of improving a cardiac output, comprising administering to a mammal in need thereof an effective amount of a urolithin; wherein the effective amount of the urolithin is about 10 mg/kg to about 30 mg/kg.

12. The method of claim 11, wherein the cardiac output is heart rate.

13. The method of claim 11, wherein the cardiac output is stroke volume.

14. The method of claim 11, wherein the cardiac output is heart rate variability.

15. A method of increasing ventricular ejection fraction, comprising administering to a mammal in need thereof an effective amount of a urolithin; wherein the effective amount of the urolithin is about 10 mg/kg to about 30 mg/kg.

16. The method of claim 15, wherein left ventricular ejection fraction (LVEF) of the mammal is increased.

17. The method of claim 16, wherein LVEF of the mammal is about less than 50% prior to administration of the urolithin.

18. The method of claim 16, wherein LVEF of the mammal is about less than 40% prior to administration of the urolithin.

19. The method of claim 16, wherein LVEF of the mammal is about less than 35% prior to administration of the urolithin.

20. The method of claim 16, wherein LVEF of the mammal is about less than 30% prior to administration of the urolithin.

21. The method of claim 16, wherein LVEF of the mammal is about 15-50% prior to administration of the urolithin.

22. The method of claim 16, wherein LVEF of the mammal is about 15-40% prior to administration of the urolithin.

23. The method of claim 16, wherein LVEF of the mammal is about 15-35% prior to administration of the urolithin.

24. The method of claim 16, wherein LVEF of the mammal is about 20-45% prior to administration of the urolithin.

25. The method of claim 16, wherein LVEF of the mammal is about 40-49% prior to administration of the urolithin.

26. The method of claim 16, wherein LVEF of the mammal is about 41-49% prior to administration of the urolithin.

27. The method of any one of claims 15-26, wherein right ventricular ejection fraction (RVEF) of the mammal is increased.

28. The method of claim 27, wherein RVEF of the mammal is about less than 50% prior to administration of the urolithin.

29. The method of claim 27, wherein RVEF of the mammal is about less than 40% prior to administration of the urolithin.

30. The method of claim 27, wherein RVEF of the mammal is about less than 35% prior to administration of the urolithin.

31. The method of claim 27, wherein RVEF of the mammal is about less than 30% prior to administration of the urolithin.

32. The method of claim 27, wherein RVEF of the mammal is about 15-50% prior to administration of the urolithin.

33. The method of claim 27, wherein RVEF is about 15-40% prior to administration of the urolithin.

34. The method of claim 27, wherein RVEF is about 15-35% prior to administration of the urolithin.

35. The method of claim 27, wherein RVEF of the mammal is about 20-45% prior to administration of the urolithin.

36. The method of claim 27, wherein RVEF of the mammal is about 40-49% prior to administration of the urolithin.

37. The method of claim 27, wherein RVEF of the mammal is about 41-49% prior to administration of the urolithin.

38. The method of any one of claims 15-37, wherein heart function is improved.

39. The method of claim 38, wherein heart function with preserved ejection fraction (HFpEF) is improved.

40. The method of claim 38, wherein heart function with reduced ejection fraction (HFrEF) is improved.

41. The method of any one of claims 1-40, wherein heart systolic function is improved.

42. A method of increasing heart systolic function, comprising administering to a mammal in need thereof an effective amount of a urolithin; wherein the effective amount of the urolithin is about 10 mg/kg to about 30 mg/kg.

43. The method of claim 41 or 42, wherein heart systolic function of the mammal is increased by about 30-80%.

44. The method of claim 41 or 42, wherein heart systolic function of the mammal is increased by about 50-70%.

45. The method of claim 41 or 42, wherein heart systolic function of the mammal is increased by about 56%.

46. The method of claim 41 or 42, wherein heart systolic function of the mammal is increased by about 64%.

47. The method of any one of claims 1-46, wherein heart diastolic dysfunction of the mammal is decreased.

48. The method of claim 47, wherein heart diastolic dysfunction of the mammal is decreased by about 5-10%.

49. A method of increasing heart systolic function and skeletal muscle function, comprising administering to a mammal in need thereof an effective amount of a urolithin; wherein the effective amount of the urolithin is about 10 mg/kg to about 30 mg/kg; and the mammal is an aging mammal.

50. The method of claim 49, wherein heart systolic function of the mammal is increased by at least about 20%, 25%, 30%, or 35%.

51. The method of claim 49, wherein heart systolic function of the mammal is increased by at least about 30%.

52. The method of any one of claims 49-51, wherein skeletal muscle function of the mammal is increased by about 2-5%.

53. A method of maintaining, supporting, or improving blood circulation, comprising administering to a mammal in need thereof an effective amount of a urolithin; wherein the effective amount of the urolithin is about 10 mg/kg to about 30 mg/kg.

54. A method of maintaining, supporting, or improving heart function, comprising administering to a mammal in need thereof an effective amount of a urolithin; wherein the effective amount of the urolithin is about 10 mg/kg to about 30 mg/kg.

55. A method of maintaining, supporting, or improving heart muscle function, comprising administering to a mammal in need thereof an effective amount of a urolithin; wherein the effective amount of the urolithin is about 10 mg/kg to about 30 mg/kg.

56. The method of any one of claims 1-55, wherein heart muscular contractility is increased.

57. The method of any one of claims 1-56, wherein heart fibrosis is decreased.

58. The method of any one of claims 1-57, wherein heart mitochondrial function is improved.

59. The method of any one of claims 1-57, wherein heart mitochondrial quality is improved.

60. The method of any one of claims 1-57, wherein heart mitochondrial area is increased.

61. The method of any one of claims 1-60, wherein expression of genes linked to mitochondrial function in the heart tissue is upregulated.

62. The method of claim 61, wherein the genes are genes of the mitochondrial protein-containing complex or genes of respirasomes.

63. The method of any one of claims 1-62, wherein heart mitophagy is improved.

64. The method of any one of claims 1-63, wherein plasma ceramide levels, acylcarnitine levels, brain natriuretic peptide (BNP) levels, creatine kinase (CK) levels, C-reactive protein (CRP) levels, troponin levels, or galectin-2 levels are decreased.

65. The method of any one of claims 1-64, wherein heart rate is improved.

66. The method of any one of claims 1-65, wherein heart rate variability is increased.

67. The method of any one of claims 1-66, wherein circulation is improved.

68. The method of any one of claims 1-67, the method further comprising transplanting mitochondria to the mammal.

69. The method of any one of claims 1-68, wherein a mitochondrial injury is improved.

70. The method of any one of claims 1-69, wherein the urolithin is administered orally.

71. The method of claim 70, wherein the urolithin is administered in the form of a tablet or capsule.

72. The method of any one of claims 1-71, wherein the urolithin is administered over a period of at least about 1 month or at least about 2 months.

73. The method of any one of claims 1-72, wherein the mammal is a human.

74. The method of claim 73, wherein the human is elderly.

75. The method of claim 73, wherein the human is at least about 50 years old.

76. The method of claim 73, wherein the human is at least about 80 years old.

77. The method of any one of claims 1-76, wherein the effective amount of the urolithin is about 25 mg/kg.

78. The method of any one of claims 1-77, wherein the urolithin is selected from the group consisting of urolithin A, urolithin B, urolithin C, urolithin D, and any combination thereof.

79. The method of any one of claims 1-77, wherein the urolithin is selected from the group consisting of urolithin A, urolithin B, and a combination of urolithin A and urolithin B.

80. The method of any one of claims 1-77, wherein the urolithin is urolithin A.

81. The method of any one of claims 1-77, wherein the urolithin is urolithin B.

82. The method of any one of claims 1-77, wherein the urolithin is urolithin C.

83. The method of any one of claims 1-77, wherein the urolithin is urolithin D.

84. The method of any one of claims 1-83, further comprising conjointly administering an additional agent to the mammal.

85. The method of claim 84, wherein the additional agent is a therapeutic agent.

86. The method of claim 85, wherein the therapeutic agent is selected from the group consisting of angiotensin-converting enzyme (ACE) inhibitors, angiotensin receptor-neprilysin inhibitors (ARNi), beta-blockers, mineralocorticoid receptor antagonists (MRAs), sodium-glucose co-transporter 2 (SGLT2) inhibitors, angiotensin-2 receptor blockers (ARBs), diuretics, If channel blockers, aldosterone antagonists, aspirin, P2Y12 inhibitors, calcium channel blockers, cholesterol-lowering drugs (e.g., statins), antiarrhythmic drugs, hydralazine with nitrate, digoxin, and anticoagulants.

87. The method of claim 86, wherein the therapeutic agent is an SGLT2 inhibitor; and the SGLT2 inhibitor is dapagliflozin or empagliflozin.

88. The method of claim 84, wherein the additional agent is a nutritional supplement.

89. The method of claim 88, wherein the nutritional supplement is selected from the group consisting of fish oils, nitrate-rich beet root, nitric oxide, red yeast rice, beta-glucans, vitamin A, vitamin C, vitamin E, vitamin K, potassium salts, magnesium salts, calcium salts, iron salts, manganese salts, copper salts, zinc salts, and phosphate salts, coenzyme q10 (Coq10), carnitine, grape seed extract, and cocoa.
